(19) United States
(12) Patent Application Publication
(10) Pub. No.: US 2024/0216593 A1
(43) Pub. Date: Jul. 4, 2024

(54) WATER PRE-TREATMENT SYSTEM FOR MEDICAL DEVICE

(71) Applicant: CVS Pharmacy, Inc., WOONSOCKET, RI (US)

(72) Inventors: Gino Ciccello, Vernon Hills, IL (US); Ricardo Hernandez, Lake Villa, FL (US)

(21) Appl. No.: 18/395,003

(22) Filed: Dec. 22, 2023

Related U.S. Application Data
(60) Provisional application No. 63/436,031, filed on Dec. 29, 2022.

(51) Int. Cl. A61M 1/16

(52) U.S. Cl. CPC A61M 1/1656; A61M 2205/3327; A61M 2205/3334

(57) ABSTRACT

A system for treatment of a fluid for a medical device. The system includes a pump coupled with an inlet tube, the pump configured to receive a fluid from the inlet tube and pump the fluid. The system includes a purification device coupled with the pump configured to treat the fluid, and a reservoir configured to house fluid in the reservoir at a first pressure. The system includes a reservoir gauge, the reservoir gauge configured to determine a pressure of the fluid in the reservoir, and an outlet tube coupled with the reservoir configured to facilitate a first flow of fluid from the reservoir. Further, the system includes a relief valve coupled with the reservoir configured to control a second flow of fluid from the reservoir, where responsive to a change in the first pressure, the relief valve is configured to modify the second flow of fluid from the reservoir.

### CROSS-REFERENCE TO RELATED PATENT APPLICATION

This application claims the benefit of and priority to U.S. Provisional Application No. 63/436,031, filed on Dec. 29, 2022, the entire disclosure of which is hereby incorporated by reference herein.

### BACKGROUND

The present disclosure generally relates to a fluid treatment system for a medical device. More specifically, according to some examples, the present disclosure relates to a fluid treatment system for a dialysis machine.

### SUMMARY

At least one example relates to a system for treatment of a fluid. In some examples, the system is for treatment of a fluid for a medical device. The system includes a pump fluidly coupled with an inlet tube, the pump configured to receive a fluid from the inlet tube and pump the fluid. The system also includes a purification device fluidly coupled with the pump, the purification device configured to receive fluid from the pump and treat the fluid. The system further includes a reservoir fluidly coupled with the purification device, the reservoir configured to house fluid in the reservoir at a first pressure, and a reservoir gauge coupled with the reservoir, the reservoir gauge configured to determine pressure of the fluid in the reservoir. The system includes an outlet tube fluidly coupled with the reservoir, the outlet tube configured to facilitate a first flow of fluid from the reservoir. Further, the system includes a relief valve fluidly coupled with the reservoir, the relief valve configured to control a second flow of fluid from the reservoir, where responsive to a change in the first pressure of the fluid in the reservoir, the relief valve is configured to modify the second flow of fluid from the reservoir.

Another example relates to a medical device. In some examples, the medical device is a dialysis machine. The dialysis machine includes an actuator, the actuator configured to move a contaminated fluid. The dialysis machine also includes a dialyzer fluidly coupled with the actuator, the dialyzer configured to receive the contaminated fluid and treat the contaminated fluid with a dialysate solution, where the dialysate solution is a solution formed via mixing a fluid from a fluid treatment system. The fluid treatment system includes a pump, a purification device fluidly coupled with the pump, the purification device configured to receive fluid from the pump and treat the fluid, and a reservoir fluidly coupled with the purification device, the reservoir configured to house fluid in the reservoir at a first pressure. The fluid treatment system also includes an outlet tube fluidly coupled with the reservoir, the outlet tube configured to facilitate a first flow of fluid from the reservoir to the dialysis machine, and a relief valve fluidly coupled with the reservoir, the relief valve configured to control a second flow of fluid from the reservoir, where responsive to a change in the first pressure of the fluid in the reservoir, the relief valve is configured to modify the second flow of fluid from the reservoir.

Another example relates to a system for treatment of a fluid. In some examples, the system is for treatment of a fluid for a medical device. The system includes an inlet valve, the inlet valve configured to receive a fluid and control a first flow of fluid, and a pump fluidly coupled with the inlet valve, the pump configured to receive the first flow of fluid from the inlet valve and pump the fluid. The system also includes a purification device fluidly coupled with the pump, the purification device configured to receive fluid from the pump and treat the fluid, and a reservoir fluidly coupled with the purification device, the reservoir configured to house fluid in the reservoir at a first pressure. The system also includes a reservoir gauge coupled with the reservoir, the reservoir gauge configured to determine pressure of the fluid in the reservoir, and a relief valve fluidly coupled with the reservoir, the relief valve configured to control a second flow of fluid from the reservoir, where responsive to the reservoir gauge determining the pressure of the fluid in the reservoir is the first pressure, the inlet valve is configured to transition to a closed state and restrict the first flow of fluid to the pump.

Another example relates to a method for controlling a characteristic of a fluid. In some examples, the method is for controlling a pressure of fluid for a medical device. The method includes determining, via a reservoir gauge, a first pressure of fluid within a reservoir, and determining, via the reservoir gauge, a change in the first pressure of the fluid within the reservoir, where the change in the first pressure is responsive to a change in a first flow of fluid from the reservoir. The method also includes restricting, via a relief valve fluidly coupled with the reservoir, a second flow of fluid from the reservoir, where the second flow of fluid is restricted by an amount equal to the change in the first flow of fluid from the reservoir. The method further includes determining, via the reservoir gauge, a second pressure of the fluid within the reservoir, where a difference between the first pressure and the second pressure is less than a predetermined threshold.

Examples may include one of the following features, or any combination thereof. In some examples, the system is a water pre-treatment system and the medical device is a dialysis machine. In other examples, the change in the first pressure of the fluid in the reservoir is responsive to an increase in the first flow of fluid from the reservoir. In some examples, the change in the first pressure of the fluid in the reservoir is a decrease in the first pressure of the fluid. In other examples, responsive to the change in the first pressure of the fluid in the reservoir, the relief valve is configured to decrease the second flow of fluid from the reservoir. In some examples, the relief valve is configured to decrease the second flow of fluid from the reservoir by an amount equal to an amount of the first flow of fluid from the reservoir. In other examples, responsive to the change in the first pressure of the fluid in the reservoir, the relief valve is configured to modify the second flow of fluid from the reservoir to maintain a second pressure of the fluid in the reservoir. In some examples, the first pressure and the second pressure are the same. In other examples, the first pressure is between 62.5 pounds per square inch and 67.5 pounds per square inch, and the second pressure is less than the first pressure.

Examples may further include one of the following features, or any combination thereof. In some examples, responsive to the change in the pressure of the fluid in the reservoir, the relief valve is configured to modify the second flow of fluid from the reservoir such that the change in the first pressure does not exceed a predetermined threshold. In other examples, the predetermined threshold is between 5 pounds per square inch and 10 pounds per square inch. In some examples, responsive to the change in the first pressure of the fluid in the reservoir, the inlet tube is configured to receive a third flow of fluid. In other examples, the third flow of fluid is in an amount equal to an amount of the first flow of fluid from the reservoir. In some examples, the system further comprises an inlet valve fluidly coupled with the inlet tube, the inlet valve configured to control a third flow of fluid to the inlet tube. In other examples, responsive to the change in the first pressure of the fluid in the reservoir, the inlet valve is configured to increase the third flow of fluid to the inlet tube. In some examples, responsive to the change in the first pressure of the fluid in the reservoir, the relief valve is configured to decrease the second flow of fluid from the reservoir, and the inlet valve is configured increase the third flow of fluid to the inlet tube, wherein the decrease in the second flow of fluid from the reservoir and the increase in the third flow of fluid to the inlet tube are equal. In other examples, the pump is configured to pump the fluid at a constant flow rate. In some examples, the system further comprises a flowmeter communicably coupled with the pump, wherein the flowmeter is configured to determine a flow rate of fluid from the pump. In other examples, responsive to the flowmeter determining a flow rate of the fluid from the pump is above a threshold, the pump is configured to decrease the flow rate of the fluid from the pump. In some examples, the system further comprises a feedback tube fluidly coupled with the relief valve and the inlet tube, wherein the feedback tube is configured to facilitate movement of fluid from the relief valve to the inlet tube.

This summary is illustrative only and should not be regarded as limiting. All examples and features mentioned above can be combined in any technically possible way.

### BRIEF DESCRIPTION OF THE FIGURES

The disclosure will become more fully understood from the following detailed description, taken in conjunction with the accompanying figures, wherein like reference numerals refer to like elements, in which:

FIG. 1 depicts a medical device for use with a patient, according to an example;

FIG. 2 depicts a block diagram of a fluid treatment system for the medical device of FIG. 1, according to an example;

FIG. 3 depicts a flow diagram of a mode of operation of the fluid treatment system of FIG. 2, according to an example;

FIG. 4 depicts a flow diagram of another mode of operation of the fluid treatment system of FIG. 2, according to an example;

FIG. 5 depicts a flow diagram of another mode of operation of the fluid treatment system of FIG. 2, according to an example;

FIG. 6 depicts a flow diagram of an example implementation of the fluid treatment system of FIG. 2, according to an example.

### DETAILED DESCRIPTION

Before turning to the figures, which illustrate certain examples in detail, it should be understood that the present disclosure is not limited to the details or methodology set forth in the description or illustrated in the figures. It should also be understood that the terminology used herein is for the purpose of description only and should not be regarded as limiting.

This disclosure is generally directed to a fluid treatment system for a medical device. According to some examples, the present disclosure relates to a fluid (e.g., water) treatment (e.g., pre-treatment) system for a dialysis machine. Dialysis machines are used to remove fluid from, filter, and/or return fluid to a patient, for example to remove waste, toxins, or excess fluid (e.g., water) from the fluid (e.g., blood) of the patient. In order to remove waste or toxins from a patient's blood, dialysis machines include components that remove (i.e., filter, pull, purify, etc.) toxins and other contaminants. For example, dialysis machines can include a dialyzer, which contains a network of small tubes submerged in a dialysate solution. The dialysate solution can be a combination of purified water, electrolytes, and salt (e.g., bicarbonate, sodium, etc.). As a patient's blood moves through the tubes of the dialyzer, the surrounding dialysate solution may remove toxins or contaminants from the blood (e.g., via diffusion) to filter the blood. Further, once the patient's blood moves through the dialyzer, the filtered blood may be returned to the patient, and the used dialysate solution may be removed from the dialysis machine. Given the flow of dialysate solution into and out of the dialysis machine during operation (e.g., the filtration process), dialysis machines may require a fluid source that provides a flow of purified water to the machine (e.g., to form the dialysate solution). While various examples herein discuss fluid (e.g., water) treatment/pre-treatment systems, it should be understood that the present disclosure encompasses use of the features with any type of treatment system.

Dialysis machines can receive fluid (e.g., purified water) from an external fluid source or water pre-treatment system. In operation, the functionality of a dialysis machine may depend on one or more characteristics of the fluid the dialysis machine receives (e.g., from the pre-treatment system). For example, dialysis machines can operate at a target internal fluid pressure (e.g., 65 pounds per square inch “psi”) and may not tolerate large fluctuations in pressure, for example those caused by the pressure of fluid provided by a water pre-treatment system. More specifically, pre-treatment systems may not provide fluid to dialysis machines at a predetermined or target pressure (e.g., 65 psi), or fluid that is within a predetermined target range (e.g., 62.5 psi-67.5 psi, etc.). Rather, pre-treatment systems may provide fluid at atmospheric pressure at a varying flow rates. As such, pre-treatment systems can provide fluid to dialysis machines in a manner that results in fluctuations in machine performance, reduces operating efficiencies, and reduces overall performance of the dialysis machine.

According to various examples, the examples described herein may control the flow of fluid such that fluid delivered to a dialysis machine may be at a predetermined pressure (e.g., 65 psi), or within a predetermined pressure range (e.g., 62.5-67.5 psi). Further, in some examples, the exemplary features described herein may control the flow of fluid, such that the delivered fluid does not fluctuate more than a predetermined variance of deviation (e.g., up or down by 10 psi). Yet further, in some examples, the exemplary features described herein can control the flow of fluid such that fluid is pumped through the fluid treatment system at a constant flow rate or speed, for example to reduce fluctuations in the fluid characteristics (e.g., pressure, flow rate, etc.) delivered to a dialysis machine. The exemplary components that facilitate the exemplary features described herein are described in further detail below.

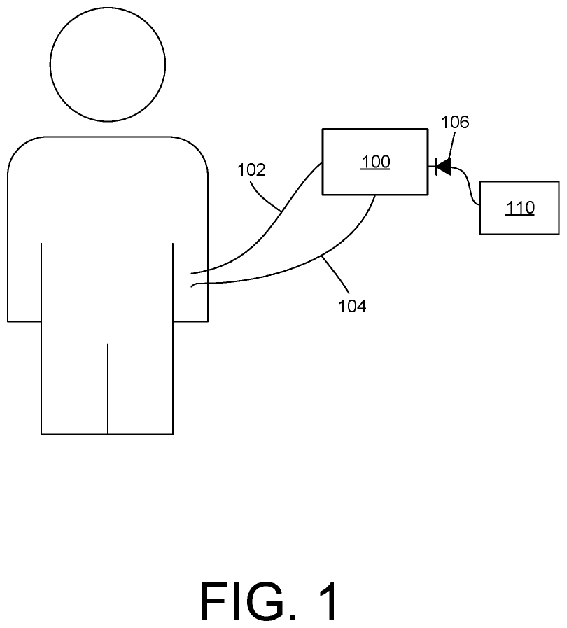

FIG. 1 depicts an example medical device for use with a patient. According to an example, the medical device is a dialysis machine 100. The dialysis machine 100 may remove fluid from, filter fluid, and/or return fluid to a patient, for example to remove waste, toxins, or excess fluid from the patient. The dialysis machine 100 is shown to include at least one tube, which may selectively couple a patient and facilitate the movement of fluid (e.g., blood) from/to the patient. For example, the dialysis machine 100 may include a first tube 102 and a second tube 104. The first tube 102 may be coupled with a patient, for example an artery of the patient. The second tube 104 may also be coupled with a patient, for example a vein of the patient. In other examples, the dialysis machine 100 may be or include any of a variety of devices, for example a hemodialysis device, a peritoneal dialysis device, a hemofiltration device, or another medical device. In some examples, features of the present disclosure may be used with devices other than medical devices, such as to assist with maintaining a consistent pressure of fluid flowing into an industrial device.

In an example, the dialysis machine 100 includes one or more components configured to remove, filter, and/or return fluid from/to the patient. For example, the dialysis machine 100 may include a pump configured to facilitate the flow of fluid from the patient to the dialysis machine 100 (e.g., from the patient's artery via the first tube 102). In an example, the dialysis machine 100 includes a dialyzer, which may filter or remove waste, toxins, or excess fluid (e.g., water) as the fluid (e.g., blood) from the patient flows through the dialyzer. The dialyzer may also be configured to receive one or more solutions (e.g., dialysate solution), for example to facilitate the removal of waste, toxins, or excess fluid. Once fluid from the patient is filtered or treated, the pump may further be configured to facilitate the flow of the filtered fluid through the dialysis machine 100 and back to the patient (e.g., to the patient's vein via the second tube 104).

The dialysis machine 100 is also shown to include a device inlet valve 106. The device inlet valve 106 may be coupled with a fluid source, and may be configured to control the flow of a fluid to the dialysis machine 100. For example, the device inlet valve 106 may be coupled (e.g., fluidly coupled) with a fluid treatment system 110. In an example, the fluid treatment system 110 is a fluid pre-treatment system, and is configured to treat (e.g., purify, filtrate, etc.) fluid prior to delivery to the dialysis machine 100. For example, the fluid treatment system 110 may be a water pre-treatment system or a water purification system, configured to purify or filtrate water delivered to the dialysis machine 100. Further, the fluid treatment system 110 may be configured to control the flow of fluid to the dialysis machine 100. For example, the fluid treatment system 110 may be configured to control the flow of purified water to the dialysis machine 100 (e.g., to form dialysate solution for use in the dialyzer). In an example, the fluid treatment system 110 is configured to control one or more characteristics of fluid that flows to the dialysis machine 100 (e.g., pressure, flow rate, temperature, purity, clarity, pH, etc.). In an example, the fluid treatment system 110 is integrated with the dialysis machine 100. In other examples, the fluid treatment system 110 is a discrete component fluidly coupled with the dialysis machine 100.

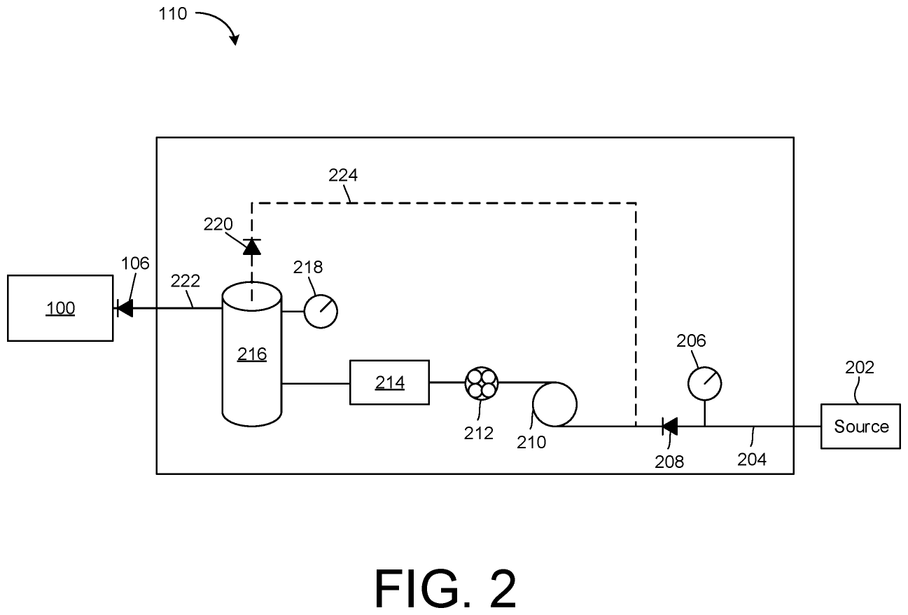

FIG. 2 depicts an example block diagram of the fluid treatment system 110. As discussed above, the fluid treatment system 110 may be configured to control the flow of fluid (e.g., purified water, purified fluid, etc.) to the dialysis machine 100, for example to be used in a dialysate solution. In an example, the fluid treatment system 110 is configured to control the flow of fluid (e.g., within the fluid treatment system 110) such that fluid is delivered to the dialysis machine 100 at a predetermined pressure, for example 65 psi. The fluid treatment system 110 may control the flow of fluid (e.g., within the fluid treatment system 110) such that the fluid delivered to the dialysis machine 100 does not fluctuate above a predetermined deviation, for example 10 psi. In some examples, the fluid treatment system 110 is configured to control the flow of fluid such that fluid is delivered to the dialysis machine at another predetermined pressure (e.g., 45, 50, 55, 60, 70, 75, 80, 90, 100, etc. psi) and/or such that the fluid delivered to the dialysis machine 100 does not fluctuate above another predetermined deviation (e.g., 4.5, 5, 7.5, 8, 9, etc. psi). The fluid treatment system 110 may also control the flow of fluid (e.g., within the fluid treatment system 110) such that the fluid is pumped through the fluid treatment system 110 at a constant flow rate (e.g., in a standby mode, a bypass mode, etc.). Further, the fluid treatment system 110 may also be configured to control other characteristics of fluid within the fluid treatment system 110 and/or the fluid delivered to the dialysis machine 100, as discussed below.

The fluid treatment system 110 is shown as coupled with a fluid source 202. The fluid source 202 may be a reservoir, a container, a tank, or other suitable vessel to selectively house a fluid. In an example, the fluid source 202 houses a fluid (e.g., water) or solution, which may be modified (e.g., purified, filtered, treated, etc.) via the fluid treatment system 110, and/or otherwise utilized during operation of the dialysis machine 100 (e.g., as part of a dialysate solution, etc.). In an example, the fluid source 202 is a discrete component fluidly coupled with the fluid treatment system 110. However, in other examples the fluid source 202 is integrated with the fluid treatment system 110 and/or the dialysis machine 100.

The fluid treatment system 110 is shown to include an inlet tube 204. The inlet tube 204 may be coupled (e.g., fluidly coupled) with the fluid source 202, and may facilitate movement of fluid from the fluid source 202 to one or more components of the fluid treatment system 110 (e.g., via an inlet of the fluid treatment system 110). As described herein, the term “tube” may describe or include any component or structure capable of facilitating movement of a fluid, substance, or other material from a first location to a second location. The inlet tube 204 may be formed of polyvinyl chloride (PVC), polyethylene, thermoplastic elastomers (TPE), nylon and silicone, or another suitable material, and may have features (e.g., thickness, diameter, length, etc.) suitable to facilitate movement of fluid to the fluid treatment system 110.

The fluid treatment system 110 may also include a gauge 206. The gauge 206 may be coupled (e.g., fluidly coupled) with the inlet tube 204, and may be configured to determine a characteristic of the fluid in the inlet tube 204. For example, the gauge 206 may be a pressure gauge configured to determine a pressure (e.g., fluid pressure) in the inlet tube 204 (e.g., at the gauge 206), for example as fluid flows from the fluid source 202 to components of the fluid treatment system 110. In an example, the gauge 206 constantly determines the pressure of the fluid in the inlet tube 204 (e.g., in real-time); however, in some examples the gauge 206 periodically (e.g., at periodic intervals, above or below a threshold, etc.) or otherwise determines the pressure of the fluid in the inlet tube 204. The gauge 206 may also be configured to instantaneously (e.g., immediately, etc.) determine a change in pressure of the fluid in the inlet tube 204.

In other examples, the gauge 206 is another suitable gauge, sensor, or measuring device (e.g., flow meter, temperature sensor, salinity meter, potential of hydrogen (pH) meter, etc.) configured to determine another characteristic (e.g., flow rate, temperature, fluid purity, clarity, pH, etc.) of fluid in the inlet tube 204. The gauge 206 may be communicably coupled with one or more components of the fluid treatment system 110 (e.g., a valve, a gauge, a pump, a vent, etc.), for example to communicate the determined characteristic to components of the fluid treatment system 110. Further, the gauge 206 may also be communicably coupled with one or more devices (e.g., the dialysis machine 100, a remote computing device), and may communicate the determined characteristic to the one or more devices.

The fluid treatment system 110 is also shown to include an inlet valve 208. The inlet valve 208 may be coupled (e.g., fluidly coupled) with the inlet tube 204, and may be configured to control the flow of fluid to the fluid treatment system 110. For example, the inlet valve 208 may control the flow of fluid from the fluid source 202, through the inlet tube 204, and to components of the fluid treatment system 110 (e.g., a pump, a flow meter, a vent, etc.). In an example, the inlet valve 208 is a variable orifice valve. The inlet valve 208 (e.g., variable orifice valve) may control the flow of fluid to components of the fluid treatment system 110 based on one or more characteristics of fluid of the fluid treatment system 110 (e.g., pressure, flow rate, etc.). For example, the inlet valve 208 may control the flow of fluid based on a pressure or a flow rate of fluid being drawn from the fluid treatment system 110 (e.g., from a reservoir via the dialysis machine 100, etc.), as discussed below. Further, the inlet valve 208 may control the flow of fluid through the inlet valve 208 based on a pressure or a flow rate of the fluid from the fluid source 202 (e.g., determined via the gauge 206), a pressure or flow rate of fluid within a control loop of the fluid treatment system 110 (e.g., determined via a gauge or a flow meter, etc.), or another suitable characteristic of the fluid treatment system 110 (e.g., a position or configuration of a relief valve, a fluid temperature, purity, clarity, pH, or other suitable characteristic).

In other examples, the inlet valve 208 is another suitable valve (e.g. ball, gate, globe, butterfly, needle, check, plug, or another suitable type of valve) configured to control the flow of fluid to the fluid treatment system 110. The inlet valve 208 may also be communicably coupled with one or more components of the fluid treatment system 110 (e.g., a gauge a pump, a vent, etc.), for example to communicate fluid characteristics and/or automate control of the flow of fluid through the inlet valve 208. Further, the inlet valve 208 may also be communicably coupled with one or more devices (e.g. the dialysis machine 100, a remote computing device), for example to automate the control of the inlet valve 208.

The fluid treatment system 110 may further include a pump 210. The pump 210 may be coupled (e.g., fluidly coupled) with the inlet tube 204, and may be configured to pump fluid through the fluid treatment system 110. For example, the pump 210 may receive fluid from the fluid source 202 (e.g., via the inlet tube 204), and may pump fluid to other components of the fluid treatment system 110 (e.g., a flow meter, a purification device, etc.). The pump 210 may also be coupled (e.g., fluidly coupled) with, and/or receive fluid from, another component of the fluid treatment system 110 (e.g., a feedback tube of a feedback loop, etc.), and may be configured to pump the fluid to other components of the fluid treatment system 110. In an example, the pump 210 is configured to pump fluid at a predetermined rate (e.g., 2-L/min, etc.), or within a predetermined range of rates (e.g., 1.5-L/min-2.5-L/min, etc.). In some examples, the pump 210 is configured to pump fluid at another predetermined rate (e.g., 200-mL/min, 500-mL/min, 1-L/min, 2.5-L/min, etc.), and/or within another predetermined range of rates (e.g., 200-500-mL/min, 500-mL/min-1-L/min, 1.5-2-L/min, etc.). In other examples, the pump 210 is configured to pump fluid based on another characteristic of the fluid (e.g., fluid temperature, purity, clarity, pH, or another suitable characteristic).

In an example, the pump 210 is an occlusive-type pump. The pump 210 may be a positive-displacement pump (e.g., external gear, internal gear, three-lobe, four-lob, sliding-vane, swinging vane cam-and-piston, or another suitable positive-displacement pump), a centrifugal pump (e.g., axial, radial, mixed, single stage, multistage, single or double volute, single or double suction, radial or axial split, vertical or horizontal shaft, or another suitable centrifugal pump), an axial-flow pump, or another suitable type of pump. Like other components of the fluid treatment system 110, the pump 210 may be communicably coupled with one or more components of the fluid treatment system 110 (e.g., a gauge, a valve, a vent, etc.), and/or one or more devices (e.g., the dialysis machine 100, a remote computing device), for example to communicate information relating to the pump 210 and/or automate control of the pump 210.

The fluid treatment system 110 is shown to include a flow meter 212. The flow meter may be coupled (e.g., fluidly coupled) with the pump 210, and may be configured to determine a characteristic of the fluid pumped from the pump 210. For example, the flow meter 212 may determine a flow rate of fluid received from the pump 210. In some examples, the flow meter 212 is coupled with the pump 210 (e.g., integrated with, directly coupled with) such that the flow meter 212 receives fluid directly from the pump 210. In an example, the flow meter 212 and the pump 210 are communicably coupled, and/or form a communications loop (e.g., feedback loop, control loop, etc.). For example, responsive to the flow meter 212 determining a flow rate above or below a threshold, the pump 210 (e.g., via feedback controls, etc.) may adjust or modify operating conditions (e.g., decrease or increase speed, alter flow patterns, etc.). Further, responsive to the flow meter 212 determining a flow rate outside a predetermined range, the pump 210 (e.g. via feedback controls, etc.) may adjust or modify operating conditions (e.g., decrease or increase speed, alter flow patterns, etc.). In an example, the communications loop (e.g., control loop, feedback loop, etc.) between the pump 210 and the flow meter 212 is configured to ensure that the pump 210 operates (and the fluid pumped from the pump 210 is) at predetermined parameters (e.g., a target or predetermined speed, a target flow rate or pressure, etc.), and/or within a predetermined range of parameters (e.g., a range of operating speed, a range of flow rates or pressures, etc.).

In other examples, the flow meter 212 is another suitable gauge, sensor, or measuring device (e.g., a pressure gauge, temperature sensor, salinity meter, potential of hydrogen (pH) meter, etc.) configured to determine a characteristic (e.g., pressure, temperature, fluid purity, fluid clarity, etc.) of fluid from the pump 210. The flow meter 212 may be communicably coupled with one or more components of the fluid treatment system 110 (e.g., a valve, a gauge, a vent, etc.), for example to communicate information to components of the fluid treatment system 110. Further, the flow meter 212 may also be communicably coupled with one or more devices (e.g., the dialysis machine 100, a remote computing device), and may communicate with the one or more devices.

The fluid treatment system 110 may also include a purification device 214. The purification device 214 may be coupled (e.g., fluidly coupled) with the pump 210, and may be configured to receive fluid from the pump 210 (e.g., via a connection tube from the flow meter 212). The purification device 214 may be configured to modify a characteristic of fluid (e.g., filter, purify, etc.) received from the pump 210. For example, the purification device 214 may be a reverse osmosis purification device configured to purify fluid (e.g., water) received from the pump 210. In an example, the purification device 214 is configured to receive fluid from the pump 210, for example fluid pumped from the fluid source 202 (e.g., water) and/or fluid pumped from a feedback or control loop of the fluid treatment system 110 (e.g., purified water), as discussed below. In this regard, the purification device 214 may be an indiscriminate purification device configured to receive various fluids from the pump 210 (e.g., water, purified water, etc.) in one or more modes of operation, as discussed below. In other examples, the purification device 214 is a purification module, a purification membrane, or another suitable purification device configured to modify a characteristic of fluid (e.g., deionization module, ultraviolet treatment module, carbon block device, chlorine removal device, sediment filter or filtration device, or another suitable purification device). In an example, the purification device 214 is communicably coupled with one or more components of the fluid treatment system 110 (e.g., a valve, gauge, vent), and/or one or more devices (e.g., the dialysis machine 100, a remote computing device), for example to communicate purification information gathered from the purification device 214.

The fluid treatment system 110 is also shown to include a reservoir 216. The reservoir 216 may be coupled (e.g., fluidly coupled) with the purification device 214, and may be configured to selectively house fluid of the fluid treatment system 110. The reservoir 216 may be configured to receive fluid (e.g., purified fluid) from the purification device 214 (e.g., via a connection tube), for example as a result of the pump 210 moving fluid through the purification device 214 and to the reservoir 216. The reservoir 216 may be configured to house fluid (e.g., water, purified water, purified fluid, etc.) of the fluid treatment system 110 at a predetermined pressure, for example 65 psi, or within a predetermined pressure range, for example between 62.5 psi-67.5 psi. In other examples, the reservoir 216 is configured to house fluid at another predetermined pressure (e.g., 61, 62, 63, 64, 66, 67, 68, 69, etc. psi), or within another range of pressures (e.g., 60-65, 60.5-69.5, 61-66, 61.5-68.5, 62-67, 63-68, 64-66, etc. psi), as discussed below. The reservoir 216 may be communicably coupled with one or more components of the fluid treatment system 110, and/or one or more devices, for example to communicate information (e.g., pressure, temperature, purity, clarity, pH, etc.) relating to fluid in the reservoir 216 to the other components or devices.

The fluid treatment system 110 may include a reservoir gauge 218. The reservoir gauge 218 may be coupled (e.g., fluidly coupled) with the reservoir 216, and may be configured to determine a characteristic of the fluid in the reservoir 216. For example, the reservoir gauge 218 may be a pressure gauge configured to determine a pressure of fluid within the reservoir 216. The reservoir gauge 218 may determine a pressure of fluid in the reservoir 216 as fluid is housed in the reservoir 216, and/or as fluid flows into/out of the reservoir 216. In an example, the reservoir gauge 218 is coupled with the reservoir 216 (e.g., integrated with, directly coupled with) such that the reservoir gauge 218 may determine (e.g., instantaneously, in real-time, etc.) a change in a pressure of the fluid in the reservoir 216. The reservoir gauge 218 may be configured to constantly determine the pressure of the fluid in the reservoir 216; however, in some examples the reservoir gauge 218 is configured to periodically (e.g., at periodic intervals, above or below a threshold, etc.) or otherwise determine the fluid pressure in the reservoir 216.

In other examples, the reservoir gauge 218 is another suitable gauge, sensor, or measuring device (e.g., flow meter, temperature sensor, salinity meter, potential of hydrogen (pH) meter, etc.) configured to determine another characteristic (e.g., flow rate, temperature, fluid purity, fluid clarity, etc.) of fluid in the reservoir 216. The reservoir gauge 218 may be communicably coupled with one or more components of the fluid treatment system 110 (e.g., a pump, a valve, a vent, etc.), for example to communicate information (e.g., measurements, determined characteristics, detected changes, etc.) to components of the fluid treatment system 110. Further, the reservoir gauge 218 may also be communicably coupled with one or more devices (e.g., the dialysis machine 100, a remote computing device), and may communicate information to the one or more devices.

The fluid treatment system 110 is shown to include a relief valve 220. The relief valve 220 may be coupled (e.g., fluidly coupled) with the reservoir 216, and may be configured to control a flow of fluid from the reservoir 216. For example, the relief valve 220 may control the flow of fluid from the reservoir 216 to components of the fluid treatment system 110 (e.g., a connection tube of a control loop, etc.). Further, the relief valve 220 may control a characteristic of fluid flowing from the reservoir 216 to another device (e.g., the dialysis machine 100), as discussed below. In an example, the relief valve 220 is a variable orifice valve. The relief valve 220 (e.g., variable orifice valve) may be configured to control a flow or a characteristic (e.g., pressure) of fluid delivered to components of the fluid treatment system 110 and/or the dialysis machine 100, for example based on one or more characteristics of the fluid of the fluid treatment system 110 (e.g., pressure, flow rate, etc.). For example, the relief valve 220 may control the flow of fluid based on a pressure of the fluid in the reservoir 216 (e.g., determined via the reservoir gauge 218), a characteristic of the fluid delivered to the dialysis machine 100 (e.g., a pressure or flow rate determined via a gauge or a flow meter, etc.), or another suitable characteristic of the fluid treatment system 110 (e.g., a position or configuration of the inlet valve 208, fluid temperature, purity, clarity, pH, or another suitable characteristic).

In some examples, the relief valve 220 is coupled (e.g., fluidly coupled) with the reservoir 216 and the reservoir gauge 218. In other examples, the relief valve 220 is another suitable valve (e.g. ball, gate, globe, butterfly, needle, check, plug, or another suitable type of valve) configured to control the flow of fluid from the reservoir 216. The relief valve 220 may also be communicably coupled with one or more components of the fluid treatment system 110 (e.g., a gauge a pump, a vent, etc.), for example to communicate fluid information and/or automate control of the relief valve 220. Further, the relief valve 220 may also be communicably coupled with one or more devices (e.g. the dialysis machine 100, a remote computing device), for example to communicate fluid information and/or automate the control of the relief valve 220.

The fluid treatment system 110 may also include an outlet tube 222. The outlet tube 222 may be coupled (e.g., fluidly coupled) with the reservoir 216, and may facilitate movement of fluid from the reservoir 216. Further, the outlet tube 222 may be coupled (e.g., fluidly coupled) with the dialysis machine 100, and may facilitate movement of fluid to the dialysis machine 100. For example, the outlet tube 222 may be coupled (e.g., fluidly coupled) with the reservoir 216 at a first end and the dialysis machine 100 at a second end (e.g., via the device inlet valve 106). The outlet tube 222 may facilitate movement of fluid from the reservoir 216 to the dialysis machine 100, for example in response to the device inlet valve 106 being in an open state (e.g., a fully open state, a first partially open state, a second partially open state that is more open than the first open state, etc.). The outlet tube 222 may be formed of polyvinyl chloride (PVC), polyethylene, thermoplastic elastomers (TPE), nylon and silicone, or another suitable material, and may have characteristics (e.g., thickness, diameter, length, etc.) suitable to facilitate movement of fluid from the reservoir 216 and/or to the dialysis machine 100.

The fluid treatment system 110 is also shown to include a feedback tube 224. The feedback tube 224 may be coupled (e.g., fluidly coupled) with the reservoir 216, and may facilitate the flow of fluid from the reservoir 216 to one or more components of the fluid treatment system 110 (e.g., the inlet tube 204, the pump 210, etc.). In an example, the feedback tube 224 is coupled (e.g., fluidly coupled) with the relief valve 220, and facilitates the flow of fluid from the relief valve 220 (e.g., from the reservoir 216). Further, the feedback tube 224 may be coupled (e.g., fluidly coupled) with the inlet tube 204, and may facilitate movement of fluid to the inlet tube 204. For example, the feedback tube 224 may be coupled (e.g., fluidly coupled) with the reservoir 216 at a first end (e.g., via the relief valve 220) and the inlet tube 204 at a second end. The feedback tube 224 may facilitate movement of fluid from the reservoir 216 to the inlet tube 204, for example in response to the device inlet valve 106 being in a closed state (e.g., a fully closed state, a first partially closed state, a second partially closed state that is more closed than the first closed state, etc.). As shown in FIG. 2, the feedback tube 224 may be coupled with the inlet tube 204 after the inlet valve 208 (e.g., relative to the flow of fluid from the fluid source 202, through the inlet valve 208, and to the pump 210). In this regard, the feedback tube 224 may be coupled with the inlet tube 204 at a location to form a loop (e.g., a feedback loop, a standby loop, etc.), such that the pump 210 may pump fluid through the loop when the inlet valve 208 is in a closed state (e.g., a fully closed state, etc.), as discussed below. The feedback tube 224 may be formed of polyvinyl chloride (PVC), polyethylene, thermoplastic elastomers (TPE), nylon and silicone, or another suitable material, and may have characteristics (e.g., thickness, diameter, length, etc.) suitable to facilitate movement of fluid into the fluid treatment system 110.

In some examples, the fluid treatment system 110 includes additional, fewer, and/or different working components. For example, the fluid treatment system 110 may include a valve communicably coupled (e.g., fluidly coupled) with the feedback tube 224. The valve may be a check valve, for example to permit and/or limit movement of a fluid through the feedback tube 224 (e.g., limit movement of fluid back through the feedback tube 224 toward the reservoir 216, etc.). In other examples, the fluid treatment system 110 includes a vent tube communicably coupled (e.g., fluidly coupled) with the reservoir 216. The vent tube may be fluidly coupled with the reservoir 216, for example to facilitate movement of fluid (e.g., air, gas, liquid, etc.) from the reservoir 216 and/or the fluid treatment system 110. In some examples, the vent tube facilitates movement of fluid from (e.g., out of, away from, etc.) the reservoir 216 and/or the fluid treatment system 110, for example to prepare (e.g., prime) the components of the fluid treatment system 110 (e.g., the tubes, reservoir 216, etc.) for one or more modes of operation, as discussed below. The vent tube may also include one or more filters (e.g., a high-efficiency particulate absorbing filter or “HEPA” filter, etc.), sensors, and/or valves. For example, the vent tube may include a HEPA filter to facilitate movement of fluid having a predetermined characteristic (e.g., clean air, etc.) to the reservoir 216 and/or the fluid treatment system 110. The vent tube may also include a sensor, for example to detect when the fluid within the reservoir 216 and/or the fluid treatment system 110 has reached a predetermined characteristic (e.g., level, amount, pressure, etc.). The vent tube may further include a valve, for example to facilitate and/or limit movement of fluid through the tube (e.g., limit movement of fluid through the vent line when the sensor detects the fluid reaches the predetermined characteristic).

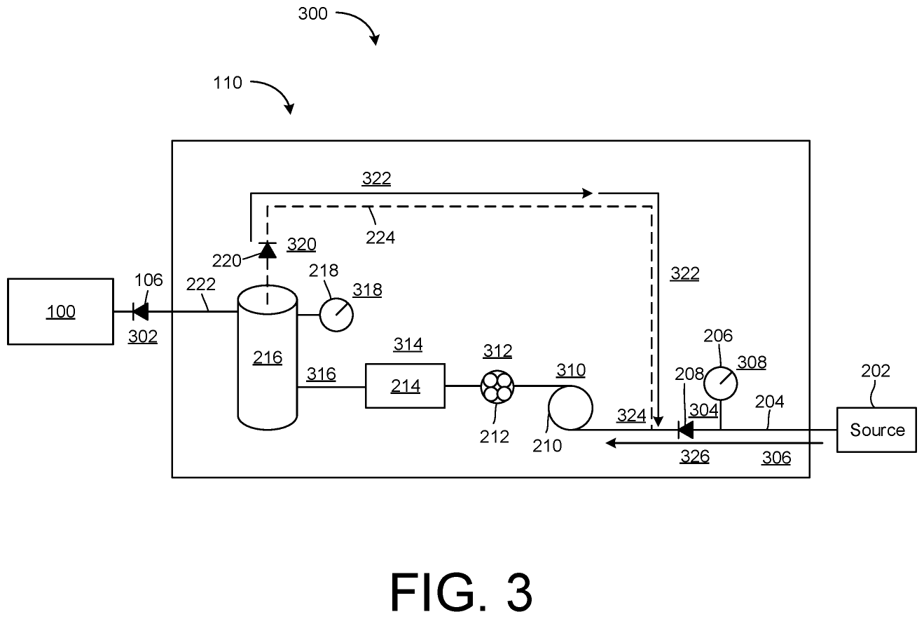

FIG. 3 depicts a flow diagram of a first mode 300 of operation of the fluid treatment system 110. Prior to, or concurrent with, entering the first mode 300, the fluid treatment system 110 may be coupled with the dialysis machine 100 (as shown in FIG. 3). According to an example, the first mode 300 is a startup mode.

At step 302, the device inlet valve 106 may be in a first state, for example a fully closed state. At step 304, the inlet valve 208 may be in a first state, for example an open state (e.g., a fully open state, a first partially open state, a second partially open state that is more open than the first open state, a non-closed, un-restrained, un-restricted, etc. state). In an example, step 302 and 304 occur simultaneously or substantially simultaneously. In some examples, step 302 and/or 304 occur concurrent with the fluid treatment system 110 being coupled with the dialysis machine 100. In some examples, step 302 and step 304 occur in sequence, vice-versa, or in a different order.

At step 306, fluid from the fluid source 202 may flow through the inlet tube 204, through the inlet valve 208, and toward other components of the fluid treatment system 110. For example, the fluid source 202 may include an actuator (e.g., a pump, motor, etc.) to move or push fluid from the fluid source 202, through the inlet tube 204, and to the inlet valve 208. In some examples, the pump 210 may draw (e.g., pull) fluid from the fluid source 202, through the inlet tube 204, and toward other components of the fluid treatment system 110 (e.g., the inlet valve 208, the pump 210, etc.). As fluid flows from the fluid source 202, through the inlet tube 204, and to the inlet valve 208, the gauge 206 may determine a characteristic of the fluid in the inlet tube 204 at step 308. In an example, the gauge 206 determines a pressure of fluid in the inlet tube 204; however, in other examples, the gauge 206 determines another suitable characteristic of the fluid (e.g., flow rate, temperature, purity, clarity, pH, etc.). The gauge 206 may communicate the determined characteristic (e.g., pressure, etc.) to other components of the fluid treatment system 110 (e.g., the inlet valve 208, the relief valve 220, etc.) and/or one or more devices (e.g., the dialysis machine 100, a remote computing device, etc.).

At step 310, the pump 210 may receive fluid from the inlet tube 204, and the pump 210 may pump the fluid toward other components of the fluid treatment system 110. For example, at step 310 the pump 210 may pump fluid toward the flow meter 212 (e.g., via a connection tube). The pump 210 may move (e.g., pump) the fluid at a predetermined speed (e.g., constant speed), or within a predetermined range of speeds. At step 312, the flow meter 212 may receive fluid from the pump 210, and may determine a characteristic of the fluid. For example, the flow meter 212 may determine a flow rate of the fluid as the fluid moves through the flow meter 212. The flow meter 212 may also communicate the determined characteristic (e.g., flow rate, etc.) to other components of the fluid treatment system 110 (e.g., the relief valve 220, the inlet valve 208, etc.) and/or to one or more devices (e.g., the dialysis machine 100, a remote computing device, etc.).

At step 314, the purification device 214 may receive fluid, and may modify a characteristic of the fluid. For example, the purification device 214 may receive fluid from the flow meter 212 (e.g., via a connection tube, via propulsion from the pump 210), and the purification device 214 may purify or filtrate the fluid (e.g., via reverse osmosis, deionization, etc.). The purification device 214 may also communicate purification information relating to the fluid and/or the purification process to other components of the fluid treatment system 110 and/or to one or more devices (e.g., the dialysis machine 100, a remote computing device, etc.).

At step 316, the reservoir 216 may receive fluid, and may selectively house the fluid. For example, the reservoir 216 may receive fluid (e.g., purified fluid) from the purification device 214 (e.g., via a connection tube, via propulsion from the pump 210, etc.). The reservoir 216 may house the fluid, or a portion of the fluid, at a pressure. For example, as fluid is received and housed by the reservoir 216, the reservoir gauge 218 may determine a characteristic of the fluid in the reservoir 216 at step 318. In an example, the reservoir gauge 218 determines a pressure of the fluid in the reservoir 216; however, in other examples the reservoir gauge 218 determines another suitable characteristic of the fluid in the reservoir 216 (e.g., temperature, flow rate, purity, clarity, pH, etc.). The reservoir gauge 218 may communicate the determined characteristic (e.g., pressure, etc.) to other components of the fluid treatment system 110 (e.g., the relief valve 220, the inlet valve 208) and/or one or more devices (e.g., the dialysis machine 100, a remote computing device). The reservoir gauge 218 may also determine and/or communicate the determined characteristic constantly, in real-time, or at another suitable interval (e.g., periodic interval, above or below a threshold, etc.).

As the reservoir 216 receives fluid, the reservoir 216 may fill such that a first portion of the fluid flows toward the outlet tube 222 (e.g., to the device inlet valve 106, toward the dialysis machine 100). Further, the reservoir 216 may fill such that a second portion of the fluid flows toward the relief valve 220 (e.g., via a connection tube). In an example, the reservoir 216 receives fluid (e.g., fills), such that the reservoir 216 fills and/or houses fluid at a predetermined pressure (e.g., 60 psi, 65 psi, 70 psi, etc.), or within a predetermined range of pressures (e.g., between 60-65 psi, 62.5-67.5 psi, 65-70 psi, etc.).

At step 320, the relief valve 220 may be in a first state, for example an open state (e.g., a fully open state, a first partially open state, a second partially open state that is more open than the first open state, etc.). In an example, step 320 occurs simultaneously or substantially simultaneously with step 302 and/or step 304. In some examples, step 320 occurs concurrently with the fluid treatment system 110 being coupled with the dialysis machine 100. In yet other examples, step 320 occurs in sequence with step 302 and/or 304, or in another order relative to any of the previously described steps.

At step 322, fluid from the reservoir 216 may flow through the relief valve 220, and toward other components of the fluid treatment system 110. For example, fluid from the reservoir 216 may flow through the relief valve 220, and to the feedback tube 224. In an example, a pressure gradient between the reservoir 216 and the feedback tube 224 (e.g., via an influx of fluid into the reservoir 216) may cause displacement of fluid from the reservoir 216 to the feedback tube 224. In some examples, the pump 210 may move (e.g., pump) fluid through the reservoir 216 and to the feedback tube 224. Further, at step 322 fluid may flow through the feedback tube 224, and to other components of the fluid treatment system 110 (e.g., the inlet tube 204).

At step 324, the inlet tube 204 may receive fluid from the feedback tube 224. The inlet tube 204 may receive fluid from the feedback tube 224, and may direct the fluid to other components of the fluid treatment system 110. For example, the inlet tube 204 may direct fluid (e.g., from the feedback tube 224) toward the pump 210, as discussed above with regard to step 310. With fluid flowing from the reservoir 216 to the pump 210 (e.g., via the feedback tube 224, the inlet tube 204, etc.) at step 324, components of the fluid treatment system 110 may determine one or more characteristics of the fluid treatment system 110. For example, the reservoir gauge 218 may determine a pressure of fluid in the reservoir 216, the flow meter 212 may determine a flow rate of fluid flowing from the pump 210, or another suitable characteristic may be determined (e.g., fluid temperature, purity, clarity, pH, etc.).

When the one or more characteristics of the fluid treatment system 110 are determined to be at or within a suitable range (e.g., predetermined target value, predetermined range, etc.), the inlet valve 208 may be transitioned to a second state, for example a closed state (e.g., a fully closed state, etc.) at step 326. For example, when the fluid pressure within the fluid treatment system 110 is determined to be at or within a suitable range (e.g., fluid pressure in the reservoir 216 is at 65 psi, between 62.5-67.5 psi, etc.), the inlet valve 208 may be transitioned to the second state (e.g., a fully closed state, etc.). With the inlet valve 208 in the second state, components of the fluid treatment system 110 may form a loop (e.g., a feedback loop, a standby loop, etc.), such that fluid may circulate through the fluid treatment system 110 under specific parameters (e.g., pressure, flow rate, etc.).

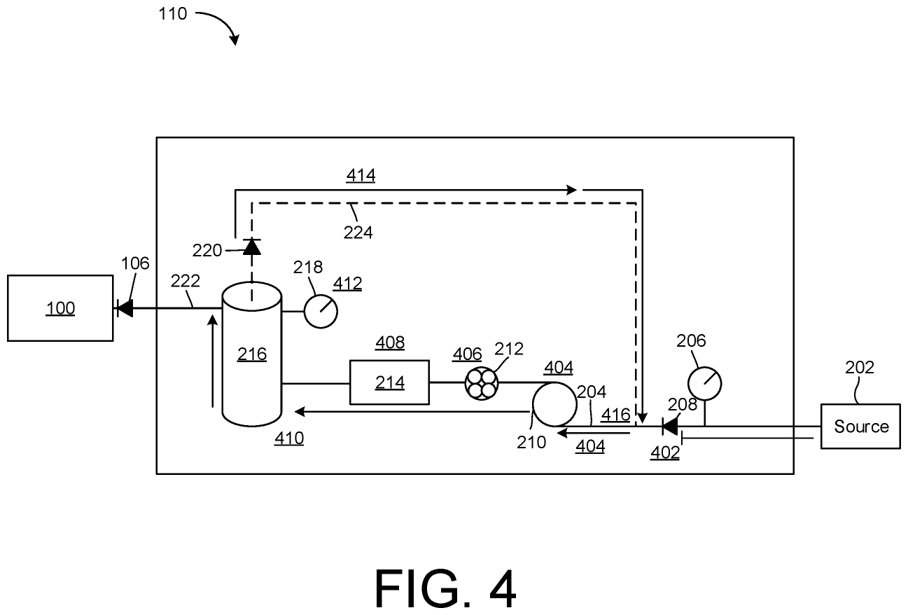

FIG. 4 depicts a flow diagram of a second mode 400 of operation of the fluid treatment system 110. Prior to, or concurrent with, entering the second mode 400, the fluid treatment system 110 may have operated in the first mode 300. In this regard, in the second mode 400 the fluid treatment system 110 may operate with a baseline amount of fluid in the fluid treatment system 110, such that one or more characteristics of the fluid treatment system 110 (e.g., pressure, flow rate, temperature, purity, clarity, pH, etc.) are at or within a suitable range (e.g., 65 psi, between 62.5-67.5 psi, etc.), as discussed above. According to an example, the second mode 400 is a standby mode.

At step 402, the inlet valve 208 may be in the second state, for example a closed state (e.g., a fully closed state, etc.), as discussed above with regard to step 326. With the inlet valve 208 in the second state, the inlet valve 208 may prevent movement of fluid from the fluid source 202 to components of the fluid treatment system 110 (e.g., the pump 210, the reservoir 216, etc.). In this regard, the inlet valve 208 may prevent introduction of new fluid to the fluid treatment system 110; thus, resulting in the existing fluid in the fluid treatment system 110 forming a loop (e.g., a closed loop, feedback loop, standby loop, etc.), as discussed above.

At step 404, fluid (e.g., from the feedback tube 224, discussed below with regard to step 416) may flow through the inlet tube 204, and toward other components of the fluid treatment system 110. For example, fluid may flow through the inlet tube 204, and toward the pump 210. In an example, the pump 210 may move (e.g., draw, pull, push, pump, etc.) fluid through the inlet tube 204, and to/through other components of the fluid treatment system 110, as discussed below.

At step 406, the pump 210 may receive fluid from the inlet tube 204, and the pump 210 may pump the fluid toward other components of the fluid treatment system 110 (e.g., the flow meter 212). In an example, the pump 210 operates at a predetermined (e.g., constant) operating speed, such that the pump 210 may move (e.g., pump) fluid at a constant speed or flow rate. For example, with the inlet valve 208 in the second state (e.g., a fully closed state, etc.), preventing introduction of fluid into the fluid treatment system 110, the existing fluid in the fluid treatment system 110 may form a loop (e.g., standby loop). The fluid in the loop (e.g., standby loop) may have one or more characteristics or properties (e.g., pressure, flow rate, purity, pH, etc.), such that the pump 210 may operate under specified operating conditions (e.g., constant speed, power, output, etc.).

Further, at step 406 the flow meter 212 may receive fluid from the pump 210, and may determine a characteristic of the fluid (e.g., a flow rate, etc.). As discussed above, the flow meter 212 and the pump 210 may be communicably coupled and form a communications loop (e.g., control loop, feedback loop, etc.). In this regard, if the flow meter 212 determines a fluid characteristic (e.g., flow rate) above or below a threshold, or outside a predetermined range, the pump 210 (e.g., via feedback controls, control loop controls, etc.) may adjust operating parameters (e.g., decrease or increase speed, alter flow patterns, etc.) until the fluid characteristic is at or within the predetermined parameters. In an example, the communications loop between the flow meter 212 and the pump 210 ensures that the pump 210 operates and/or pumps fluid at, or within a range, of specified operating conditions (e.g., speed, flow rate, etc.).

At step 408, the purification device 214 may receive fluid, and/or may modify a characteristic of the fluid. For example, the purification device 214 may receive fluid from the flow meter 212 (e.g., via a connection tube, via propulsion from the pump 210), and the purification device 214 may purify or filtrate the fluid (e.g., via reverse osmosis, deionization, etc.). In an example, the purification device 214 receives fluid from the flow meter 212 (e.g., via the pump 210), for example fluid from the inlet tube 204 (discussed above at step 404) and the feedback tube 224 (discussed below at step 416). In this regard, in the second mode 400 the purification device 214 may receive fluid (e.g., purified water) that was previously modified or treated by the purification device 214 (e.g., as discussed in FIG. 3, via the feedback or standby loop). As such, in the second mode 400 the purification device may be indiscriminate, and may be configured to permit fluid to flow through the purification device 214 without additionally modifying (e.g., filtering, treating, etc.) the fluid.

At step 410, the reservoir 216 may receive fluid, and may selectively house the fluid or a portion thereof. For example, the reservoir 216 may receive fluid (e.g., purified fluid) from the purification device 214 (e.g., via a connection tube, via propulsion from the pump 210, etc.), and may house the fluid, or a portion of the fluid. The reservoir 216 may house the fluid, or a portion of the fluid, at a pressure. In an example, the reservoir 216 receives fluid (e.g., fills) such that the reservoir 216 fills and/or houses fluid at a predetermined pressure (e.g., 60 psi, 65 psi, 70 psi, etc.), or within a predetermined pressure range (e.g., between 60-65 psi, 62.5-67.5 psi, 65-70 psi, etc.). At step 412, as fluid is received and housed by the reservoir 216, the reservoir gauge 218 may determine a pressure of the fluid in the reservoir 216. The reservoir gauge 218 may communicate the determined pressure to other components of the fluid treatment system 110 and/or one or more devices (e.g., the dialysis machine 100).

Further, as the reservoir 216 receives fluid, the reservoir 216 may fill such that a portion of the fluid flows toward the relief valve 220 (e.g., via a connection tube). With the relief valve 220 in the first state (e.g., a fully open state, a first partially open state, a second partially open state that is more open than the first open state, etc.) fluid from the reservoir 216 may flow through the relief valve 220, and toward other components of the fluid treatment system 110 at step 414. For example, fluid from the reservoir 216 may flow through the relief valve 220, and to the feedback tube 224. In an example, the pump 210 may move (e.g., pump) fluid through the reservoir 216 and to the feedback tube 224. Further, at step 414 fluid may flow through the feedback tube 224, and to other components of the fluid treatment system 110 (e.g., the inlet tube 204).

At step 416, the inlet tube 204 may receive fluid from the feedback tube 224. The inlet tube 204 may receive fluid from the feedback tube 224, and may direct the fluid back to other components of the fluid treatment system 110. For example, the inlet tube 204 may direct fluid (e.g., from the feedback tube 224) back toward the pump 210, as discussed above with regard to step 404. In this regard, following step 416, step 404 may be repeated. One or more steps of the second mode 400 described above may also be repeated or implemented, such that one or more characteristics of the fluid treatment system 110 (e.g., pressure, flow rate, temperature, purity profiles, etc.) may remain at or within a suitable range in the second mode 400.

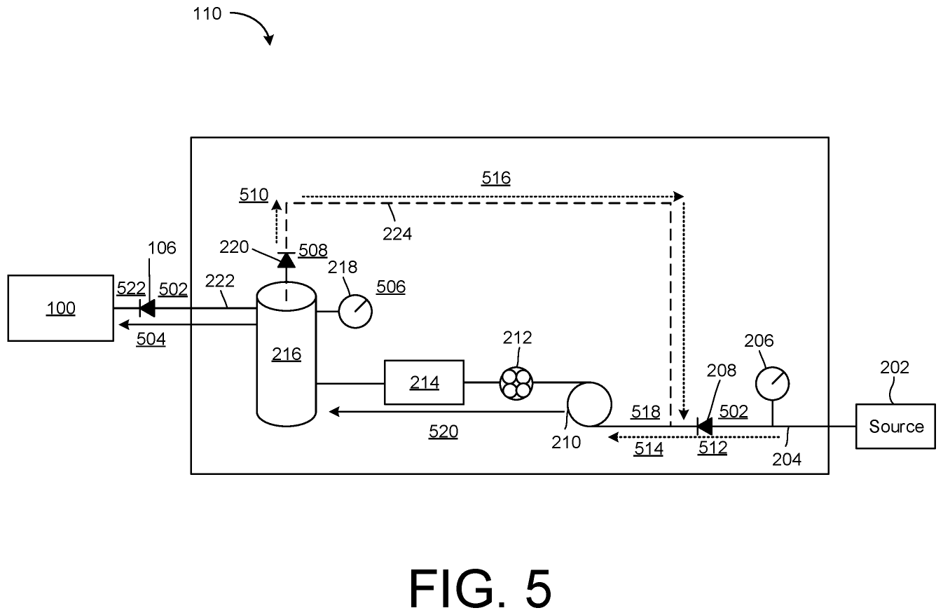

FIG. 5 depicts a flow diagram of a third mode 500 of operation of the fluid treatment system 110. Prior to, or concurrent with, entering the third mode 500, the fluid treatment system 110 may have operated in the first mode 300 and/or the second mode 400. In this regard, in the third mode 500 the fluid treatment system 110 may operate with a baseline amount of fluid in the fluid treatment system 110, such that one or more characteristics of the fluid treatment system 110 (e.g., pressure, flow rate, temperature, purity, clarity, pH, etc.) are at or within a suitable range (e.g., 65 psi, between 62.5-67.5 psi, etc.), as discussed above. According to an example, the third mode 500 is a production mode.

At step 502, the inlet valve 208 may be in the second state, for example a closed state (e.g., a fully closed state, etc.), as discussed above with regard to step 326 of FIG. 3 and/or step 402 of FIG. 4. Further, at step 502 the device inlet valve 106 may be in the first state, for example a closed state (e.g., a fully closed state, etc.), as discussed above with regard to step 302 of FIG. 3. Yet further, at step 502 components of the fluid treatment system 110 may determine one or more characteristics of the fluid treatment system 110. For example, the reservoir gauge 218 may determine a pressure of fluid in the reservoir 216, the flow meter 212 may determine a flow rate of fluid flowing from the pump 210, the gauge 206 may determine a pressure of fluid in the inlet tube 204 (e.g., at the inlet valve 208), and/or another suitable characteristic of the fluid treatment system 110 may be determined (e.g., valve configuration, fluid temperature, purity, clarity, pH, etc.). In an example, at step 502 the fluid in the reservoir 216 is determined to be at a predetermined pressure (e.g., 60 psi, 65 psi, 70 psi, etc.), or within a predetermined pressure range (e.g., between 60-65 psi, 62.5-67.5 psi, 65-70 psi, etc.).

At step 504, the device inlet valve 106 is transitioned to a second state, for example an open or non-closed state (e.g., a fully open state, a first partially open state, a second partially open state that is more open than the first open state, etc.). With the device inlet valve 106 in the second state, fluid from the reservoir 216 may flow from the reservoir 216, through the outlet tube 222, through the device inlet valve 106, and to the dialysis machine 100. In some examples, the dialysis machine 100 includes an actuator (e.g., a pump) to pull or draw fluid from the reservoir 216 (e.g., to the dialysis machine 100). In other examples, a pressure gradient between the reservoir 216 and the dialysis machine 100 (e.g., via a difference in pressure between fluid housed in the reservoir 216 and an open connection tube proximate to the device inlet valve 106) causes a displacement of fluid from the reservoir 216 to the dialysis machine 100.

At step 506, the reservoir gauge 218 may determine (e.g., detect) a change or deviation in a fluid characteristic in the reservoir 216. For example, the reservoir gauge 218 may detect a change (e.g., decrease) in pressure of fluid in the reservoir 216 (e.g., as a result of a first flow of fluid from the reservoir 216 to the dialysis machine 100). In an example, step 506 occurs instantaneously, simultaneously, or substantially simultaneously with step 504. For example, step 506 and step 504 occur within 0.0001, 0.001, 0.01, 0.1, seconds, or with another substantially simultaneous time period. In some examples, step 506 occurs immediately subsequent to step 504. In an example, the reservoir gauge 218 communicates the detected change in pressure to other components of the fluid treatment system 110 (e.g., in real-time, instantaneously, simultaneously, substantially simultaneously, immediately subsequent to, etc.). For example, the reservoir gauge 218 may communicate the change in fluid pressure to the relief valve 220, the inlet valve 208, and/or the pump 210. Further, the reservoir gauge 218 may communicate the change to another device (e.g., the dialysis machine 100, remote device, etc.).

At steps 508-510, the relief valve 220 may alter the flow of fluid through the relief valve 220. For example, responsive to receiving an indication of a change in fluid pressure within the reservoir 216, the relief valve 220 may transition to a response state at step 508 (e.g., a first partially closed state, a second partially closed state that is more closed than the first closed state, etc.). In the response state, the relief valve 220 may alter or reduce the size or configuration of an aperture of the relief valve 220. Further, the relief valve 220 may alter (e.g., change, limit, reduce, etc.) the flow of fluid from the reservoir 216 through the relief valve 220 at step 510. For example, responsive to a change in fluid pressure in the reservoir 216, the relief valve 220 may alter (e.g., change, reduce, limit, etc.) a second flow of fluid from the reservoir 216 through the relief valve 220 (e.g., at step 510). According to an example, the relief valve 220 reduces the fluid flow through the relief valve 220 by an amount proportionate to the amount of fluid drawn from the reservoir 216 (e.g., by the dialysis machine 100). For example, responsive to a first flow of fluid being drawn by the dialysis machine 100 (e.g., 1-L/min), the relief valve 220 may reduce the second flow of fluid through the relief valve 220 (e.g., 2-L/min of fluid initially moving through the relief valve 220) by the amount of fluid in the first flow (e.g., 1-L/min), as shown at step 510. In an example, step 508 and step 510 occur instantaneously, simultaneously, or substantially simultaneously with step 504 and/or step 506. In some examples, step 508 and step 510 occur immediately subsequent to step 504 and/or step 506.

According to an example, the proportionate relationship between the amount of fluid drawn from the reservoir 216 (e.g., via the dialysis machine 100), and the reduction in fluid flow through the relief valve 220, maintains the pressure within the reservoir 216 and/or limits the magnitude of pressure change within the reservoir 216. For example, following the device inlet valve 106 transitioning to the second state at step 504, the device inlet valve 106 may receive a flow of fluid from the reservoir 216 (e.g., 1-L/min). Immediately following, the reservoir gauge 218 may detect a drop in fluid pressure (e.g., 2 psi) within the reservoir 216 (e.g., from 65 psi to 63 psi) at step 506. Responsive to detecting the change in pressure in the reservoir 216, the relief valve 220 may immediately transition to the response state at step 508. In the response state, the relief valve 220 may reduce the flow of fluid through the relief valve 220, for example by an amount proportionate to the amount of fluid flowing through the device inlet valve 106 (e.g., 1-L/min reduced flow) at step 510. This reduction in fluid flow through the relief valve 220, proportionate to the amount of fluid being drawn by the dialysis machine 100 (e.g., via the device inlet valve 106), may cause an equilibrium effect that maintains or sustains a fluid pressure within the reservoir 216 (e.g., at 63 psi) and/or limits the magnitude of the pressure drop within the reservoir 216 (e.g., 2 psi).

As discussed above, in some examples the relief valve 220 is a variable orifice relief valve. In this regard, the relief valve 220 may automatically (e.g., instantaneously, immediately, etc.) transition to the response state and/or reduce the flow of fluid through the relief valve 220 by an amount proportionate to the amount of fluid drawn from the reservoir 216 (e.g., by the dialysis machine 100). Thus, the relief valve 220 may reduce the flow of fluid through the relief valve 220 within a time interval such that the change in pressure within the reservoir 216 is negligible or minimal (e.g., 0.01, 0.1, 0.25, 0.5, 0.75, 1, etc. psi). In other examples, the relief valve 220 reduces the amount of fluid through the relief valve 220 in response to the drop in fluid pressure determined by the reservoir gauge 218, for example via an automated control loop (e.g., a communications loop, feedback loop, etc.). In other examples, the relief valve 220 modifies (e.g., reduces, increases, etc.) the flow of fluid through the relief valve 220 by another amount or proportion (e.g., 0.25, 0.5, 0.75, 1.25, 5, etc. L/min; 1.5:1, 2:1, 2.5:1, 3:1, etc. ratio), based on another fluid characteristic (e.g., pressure, temperature, purity, clarity, pH, etc.), and/or based on another characteristic of one or more components of the fluid treatment system 110.

At steps 512-514, the inlet valve 208 may alter the flow of fluid through the inlet valve 208. For example, responsive to receiving an indication of a change in fluid pressure within the reservoir 216, the inlet valve 208 may transition to a demand state at step 512 (e.g. a fully open state, a first partially open state, a second partially open state that is more open than the first open state, etc.). In the demand state, the inlet valve 208 may alter or increase the size or configuration of an aperture of the inlet valve 208. Further, the inlet valve 208 may alter (e.g., change, increase, permit, allow, etc.) the flow of fluid through the inlet valve 208 and to components of the fluid treatment system 110. For example, responsive to a change in fluid pressure within the reservoir 216, the inlet valve 208 may permit fluid to flow from the fluid source 202, through the inlet valve 208, and to components of the fluid treatment system 110 (e.g., the pump 210, the reservoir 216, etc.) at step 514. According to an example, the inlet valve 208 permits a fluid flow through the inlet valve 208 in an amount proportionate to the amount of fluid drawn from the reservoir 216 (e.g., by the dialysis machine 100). For example, responsive to a first flow of fluid drawn from the reservoir 216 by the dialysis machine 100 (e.g., 1-L/min), the inlet valve 208 may permit a flow of fluid through the inlet valve 208 (e.g., from the fluid source 202) that is proportionate to the amount of fluid in the first flow (e.g., 1-L/min), as shown in step 514. In an example, step 512 and step 514 occur instantaneously, simultaneously, or substantially simultaneously with 504, step 506, and/or steps 508-510. In some examples, step 512 and step 514 occur immediately subsequent to step 504, step 506, and/or steps 508-510.

According to an example, the proportionate relationship between the amount of fluid drawn from the reservoir 216 (e.g., via the dialysis machine 100), and the increase in fluid flow through the inlet valve 208, maintains or sustains a flow of fluid within the fluid treatment system 110 (e.g., the closed loop, the feedback loop, the standby loop, etc.). For example, at step 502 (e.g., initially) the fluid treatment system 110 may have a target flow of 2-L/min. Following the device inlet valve 106 transitioning to the second state at step 504 (e.g., an open state), the device inlet valve 106 may receive a flow of fluid from the reservoir 216 (e.g., 1-L/min). Immediately following, the reservoir gauge 218 may detect a drop in fluid pressure (e.g., 2 psi) within the reservoir 216 at step 506. Responsive to detecting the change in pressure in the reservoir 216, the inlet valve 208 may immediately transition to the demand state at step 512 (e.g., an open state, etc.). In the demand state, at step 514 the inlet valve 208 may allow fluid to flow from the fluid source 202, through the inlet valve 208, and to components of the fluid treatment system 110 (e.g., 1-L/min), for example in a fluid amount proportionate to the amount of fluid flowing through the device inlet valve 106 (e.g., 1-L/min). This increase in fluid flow through the inlet valve 208, proportionate to the amount of fluid being drawn from the reservoir 216 (e.g., via the dialysis machine 100), may cause an equilibrium effect that balances the fluid flow out of/into the fluid treatment system 110, and maintains or sustains the amount of fluid flowing in the fluid treatment system 110 (e.g., 2-L/min).

As discussed above, in some examples the inlet valve 208 is a variable orifice relief valve. In this regard, the inlet valve 208 may automatically (e.g., instantaneously, immediately, simultaneously, substantially simultaneously, etc.) transition to the demand state and/or increase the flow of fluid through the inlet valve 208 by an amount proportionate to the amount of fluid drawn from the reservoir 216 (e.g., by the dialysis machine 100). Thus, the inlet valve 208 may increase the flow of fluid through the inlet valve 208 within a time interval such that the change in fluid flow within the fluid treatment system 110 is negligible or minimal (e.g., 0.001, 0.01, 0.025, 0.05, 0.075, 0.1, etc. L/min). In other examples, the inlet valve 208 increases the flow of fluid through the inlet valve 208 in response to the drop in fluid pressure determined by the reservoir gauge 218. For example, based on a pressure at the inlet valve 208 (e.g., determined by the gauge 206), and/or based on the pressure drop determined by the reservoir gauge 218, the inlet valve 208 may open or increase the size of an aperture of the inlet valve 208. The increase in aperture size, or change in aperture configuration, of the inlet valve 208 may be proportionate to the amount of fluid drawn from the reservoir 216, the fluid pressure at the inlet valve 208, and/or the fluid pressure of the reservoir 216. Further, the increase in aperture size, or change in aperture configuration, may be such that the inlet valve 208 permits an amount of fluid (e.g., proportionate to the amount of fluid drawn from the reservoir 216) to flow through the inlet valve 208, for example via an automated control loop (e.g., a communications loop, feedback loop, etc.). In other examples, the inlet valve 208 modifies (e.g., increases, reduces, etc.) the flow of fluid through the inlet valve 208 by another amount or proportion (e.g., 0.25, 0.5, 0.75, 1.25, 5, etc. L/min; 1:1.5, 1:2, 1:2.5, 1:3, etc. ratio), based on another fluid characteristic (e.g., pressure, temperature, purity, clarity, pH, etc.), and/or based on another characteristic of one or more components of the fluid treatment system 110.

At step 516, fluid from the reservoir 216 may flow through the relief valve 220, and to components of the fluid treatment system 110. For example, at step 516 fluid from the reservoir 216 (e.g., at step 510) may flow through the relief valve 220 and to the feedback tube 224. Further, fluid may flow through the feedback tube 224, and to other components of the fluid treatment system 110 (e.g., the inlet tube 204).

At step 518, the inlet tube 204 may receive fluid from the feedback tube 224. According to an example, at step 518 the inlet tube 204 receives fluid from the feedback tube 224 and fluid from the fluid source 202 (e.g., via step 514). At step 518, the inlet tube 204 may receive a flow of fluid (e.g., via the feedback tube 224 from the reservoir 216, the fluid source 202, etc.) that is equal to a target fluid flow of the fluid treatment system 110 at step 502 (e.g., prior to the device inlet valve 106 transitioning to the second, or an open state). For example, the inlet tube 204 may receive a flow of fluid from the feedback tube 224 that is equal to a second flow of fluid from the reservoir 216 (e.g., 2-L/min of fluid initially moving through the relief valve 220) reduced by an amount proportionate to the flow of fluid being drawn from the reservoir 216 (e.g., 1-L/min drawn by the dialysis machine 100), as discussed above in steps 508-510. Further, the inlet tube 204 may receive a flow of fluid from the fluid source 202 that is proportionate to the amount of fluid being drawn from the reservoir 216 (e.g., 1-L/min drawn by the dialysis machine 100), as discussed above in steps 512-514. In this regard, the reduction in the second flow of fluid from the reservoir 216 by the relief valve 220, and the proportionate increase in fluid flow from the fluid source 202 by the inlet valve 208, may cause an equilibrium effect that maintains or sustains a fluid flow through the inlet tube 204 (e.g., the fluid treatment system 110) that is equal to the target fluid flow at step 502.

At step 520, the inlet tube 204 may facilitate movement of fluid to other components of the fluid treatment system 110. For example, the inlet tube 204 may facilitate movement of fluid to the pump 210, the flow meter 212, the purification device 214, and/or the reservoir 216, as discussed above in steps 404-410 of FIG. 4. Further, at step 520 the reservoir 216 may receive a flow of fluid (e.g., from the purification device 214, via the pump 210). In an example, the reservoir 216 receives a flow of fluid that is proportionate of an amount of fluid being drawn from the reservoir 216 (e.g., via the dialysis machine 100 at step 504). In this regard, the reservoir 216 may facilitate an equal flow of fluid out of (e.g., via the dialysis machine 100) and into (e.g., via the inlet tube 204) the reservoir 216. Further, the reservoir 216 may house and/or facilitate the flow of fluid at a predetermined pressure (e.g., 60 psi, 65 psi, 70 psi, etc.), or within a predetermined pressure range (e.g., between 60-65 psi, 62.5-67.5 psi, 65-70 psi, etc.).

At step 522, the device inlet valve 106 may be transitioned to the first state, for example a closed state (e.g., a fully closed state, a first partially closed state, a second partially closed state that is more closed than the first closed state, etc.). For example, following completion of a dialysis treatment, the device inlet valve 106 may be closed, such that the dialysis machine 100 no longer draws fluid from the reservoir 216. With the device inlet valve 106 in the first or a closed state, the reservoir gauge 218 may determine (e.g., detect) a change or deviation in a fluid characteristic in the reservoir 216. For example, the reservoir gauge 218 may detect a change (e.g., increase) in pressure of fluid in the reservoir 216 (e.g., as a result of the restricted flow of fluid from the reservoir 216 to the dialysis machine 100). Responsive to receiving an indication of a change (e.g., increase) in fluid pressure in the reservoir 216, the relief valve 220 may transition from the response state (e.g., to an open state, etc.), and may permit an increased flow of fluid through the relief valve 220 (e.g., to permit an increased flow from the reservoir 216, reduce the fluid pressure in the reservoir 216). Further, responsive to receiving an indication of a change (e.g., increase) in fluid pressure in the reservoir 216, the inlet valve may transition from the demand state (e.g., to a closed state, etc.), and may reduce the flow of fluid through the inlet valve 208 (e.g., to reduce the flow of fluid into the fluid treatment system 110 from the fluid source 202, reduce the fluid pressure within the fluid treatment system 110). As the relief valve 220 transitions from the response state (e.g., to the first or an open state), and the inlet valve 208 transitions from the demand state (e.g., to the second or a closed state), components of the treatment system may determine one or more characteristics of the fluid treatment system 110, as discussed above with regard to step 324 of FIG. 3 (e.g., fluid pressure, flow rate, etc.). When the one or more characteristics of the fluid treatment system 110 are determined to be at or within a suitable range (e.g., predetermined target value, predetermined range, etc.), the inlet valve 208 may be transitioned to the second state or a closed state (e.g., a fully closed state, etc.), such that the fluid may circulate through the fluid treatment system 110 under specific parameters (e.g., pressure, flow rate, etc.) as discussed above with regard to FIG. 3 and FIG. 4.

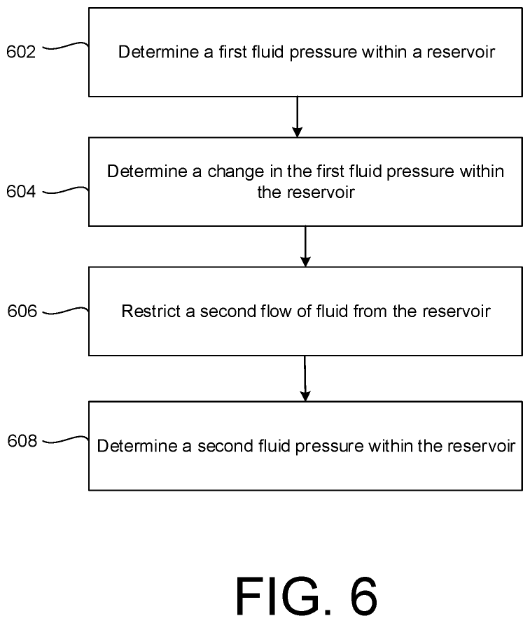

FIG. 6 depicts a flow diagram of an example implementation of a treatment system. The treatment system of FIG. 6 may be the fluid treatment system 110 described in FIGS. 1-5. Further, the example implementation of the fluid treatment system 110 described in FIG. 6 may implement any of the steps described above in FIGS. 3-5.

At step 602, a first pressure of fluid within a reservoir is determined. For example, a first pressure of the fluid within the reservoir 216 may be determined. The pressure may be of fluid housed in the reservoir 216, and/or fluid flowing into or out of the reservoir 216. In an example, the first pressure is determined via the reservoir gauge 218. In some examples, the reservoir gauge 218 is configured to communicate the determined first pressure to one or more components of the fluid treatment system 110 (e.g., the inlet valve 208, the relief valve 220, the pump 210), or another device (e.g., the dialysis machine 100, a remote computing device, etc.).

At step 604, a change in the first pressure of fluid within the reservoir is determined. For example, change in the first pressure of fluid within the reservoir 216 may be determined by the reservoir gauge 218 (e.g., instantaneously, immediately, in real-time, simultaneously, substantially simultaneously, etc.). In an example, the change in the first pressure is responsive to a change in a first flow of fluid from the reservoir 216. For example, the change in pressure may be responsive to a change (e.g., increase, etc.) in a flow of fluid from the reservoir 216 to the outlet tube 222 (e.g., the dialysis machine 100). According to an example, the change in the flow of fluid from the reservoir 216 is responsive to a change in orientation of the device inlet valve 106 (e.g., to an open state). For example, in preparation for a dialysis treatment, the device inlet valve 106 may transition to an open state (e.g., a fully open state, a first partially open state, a second partially open state that is more open than the first open state, etc.). In the open state, the device inlet valve 106 may receive (e.g., the dialysis machine 100 may draw) fluid from the reservoir 216 through the outlet tube 222. This flow of fluid through the device inlet valve 106 (e.g., via the outlet tube 222) to the dialysis machine 100, may result in the change in the first pressure of fluid within the reservoir 216.

At step 606, a second flow of fluid from the reservoir is restricted. For example, a second flow of fluid from the reservoir 216, through the relief valve 220, may be restricted. In an example, responsive to an indication of the change in pressure of fluid within the reservoir 216 (e.g., via the reservoir gauge 218 at step 604), the relief valve 220 may transition to a response state (e.g., automatically, instantaneously, immediately, simultaneously, substantially simultaneously, etc.). In the response state, the relief valve may alter or change (e.g., reduce) the flow of fluid from the reservoir 216 through the relief valve 220. According to an example, the relief valve 220 reduces the flow of fluid through the relief valve 200 by an amount that is proportionate to the amount of fluid drawn from the reservoir 216 (e.g., through the outlet tube 222 via the dialysis machine 100). For example, the relief valve 220 may reduce or restrict the second flow of from the reservoir 216 through the relief valve 220 by an amount that is equal to the change in the first flow of fluid from the reservoir 216 (e.g., to the outlet tube 222 via the dialysis machine 100).

At step 608, a second pressure of the fluid within the reservoir is determined. For example, a second pressure of the fluid within the reservoir 216 may be determined (e.g., via the reservoir gauge 218). According to an example, the difference between the first pressure and the second pressure is less than a predetermined threshold (e.g., 10 psi). For example, in response to detecting a change in pressure in the reservoir 216 (e.g., at step 604), the relief valve 220 may restrict the second flow of fluid from the reservoir 216 (e.g., at step 606), for example by an amount equal to the change in the amount of fluid being drawn from the reservoir 216. This reduction in fluid flow through the relief valve 220, proportionate to the amount of fluid being drawn from the reservoir 216 (e.g., via the dialysis machine), may cause an equilibrium effect that maintains or sustains a fluid pressure within the reservoir 216 and/or limits the magnitude of the pressure drop within the reservoir 216. In an example, the first pressure of the fluid in the reservoir is 65 psi, and the second fluid pressure in the reservoir is 65 psi. In some examples, the change in pressure in the reservoir is non-zero. However, the magnitude of the changes in pressure within the reservoir 216 (e.g., difference between the first pressure and the second pressure) may be correlated to, or limited by, the controlled flow of fluid through the relief valve 220. For example, the difference between the first pressure and the second pressure (e.g., deviation threshold) may be less than 10 psi, or another suitable threshold (e.g., 0.5, 0.75, 1, 2, 3, 4, 6, 8, etc. psi).

It should be understood that any, or all, of the apparatuses, systems, and/or methods described herein may incorporate automated devices and/or systems, such that the dialysis machine 100 and/or the fluid treatment system 110 may function automatically. For example, the valves of the fluid treatment system 110 (e.g., the inlet valve 208, relief valve 220, etc.) may include automated devices/systems (e.g., sensors, timers, actuators, motors, switches, etc.), which may be configured to automatically control the position, orientation, timing, etc. of the valves. Further, the gauge 206, the pump 210, the flow meter 212, the reservoir 216, the reservoir gauge 218 may include automated devices/systems (e.g., sensors, timers, actuators, motors, switches, etc.), which may be configured to automatically control the functions of these components. Accordingly, the fluid treatment system 110 may make use of hardware, such as a controller or data processing component (e.g., processing circuit comprising a processor and memory) to carry out the automated functionality described herein.

As utilized herein with respect to numerical ranges, the terms “approximately,” “about,” “substantially,” and similar terms generally mean+/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various examples, are intended to indicate that such examples are possible examples, representations, or illustrations of possible examples (and such terms are not intended to connote that such examples are necessarily extraordinary or superlative examples).

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other examples, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one example may be incorporated or utilized with any other example disclosed herein. For example, steps of the first mode 300 of the example described in at least FIG. 3 may be incorporated in the second mode 400 or the third mode 500 of the example described in at FIG. 4 and FIG. 5, respectively. Although only one example of an element from one example that can be incorporated or utilized in another example has been described above, it should be appreciated that other elements of the various examples may be incorporated or utilized with any of the other examples disclosed herein.

A number of implementations have been described. Nevertheless, it will be understood that additional modifications may be made without departing from the scope of the inventive concepts described herein, and, accordingly, other examples are within the scope of the following claims.

What is claimed is:

1. A system for treatment of a fluid for a medical device, the system comprising:
a pump fluidly coupled with an inlet tube, the pump configured to receive a fluid from the inlet tube and pump the fluid;
a purification device fluidly coupled with the pump, the purification device configured to receive fluid from the pump and treat the fluid;
a reservoir fluidly coupled with the purification device, the reservoir configured to house fluid in the reservoir at a first pressure;
a reservoir gauge coupled with the reservoir, the reservoir gauge configured to determine pressure of the fluid in the reservoir;
an outlet tube fluidly coupled with the reservoir, the outlet tube configured to facilitate a first flow of fluid from the reservoir; and
a relief valve fluidly coupled with the reservoir, the relief valve configured to control a second flow of fluid from the reservoir,
wherein responsive to a change in the first pressure of the fluid in the reservoir, the relief valve is configured to modify the second flow of fluid from the reservoir.

2. The system for treatment of a fluid for a medical device of claim 1, wherein the system is a water pre-treatment system and the medical device is a dialysis machine.

3. The system for treatment of a fluid for a medical device of claim 1, wherein the change in the first pressure of the fluid in the reservoir is responsive to an increase in the first flow of fluid from the reservoir.

4. The system for treatment of a fluid for a medical device of claim 1, wherein the change in the first pressure of the fluid in the reservoir is a decrease in the first pressure of the fluid.

5. The system of claim 1, wherein responsive to the change in the first pressure of the fluid in the reservoir, the relief valve is configured to decrease the second flow of fluid from the reservoir.

6. The system of claim 5, wherein the relief valve is configured to decrease the second flow of fluid from the reservoir by an amount equal to an amount of the first flow of fluid from the reservoir.

7. The system of claim 1, wherein responsive to the change in the first pressure of the fluid in the reservoir, the relief valve is configured to modify the second flow of fluid from the reservoir to maintain a second pressure of the fluid in the reservoir.

8. The system of claim 7, wherein the first pressure and the second pressure are the same.

9. The system of claim 7, wherein the first pressure is between 62.5 pounds per square inch and 67.5 pounds per square inch, and the second pressure is less than the first pressure.

10. The system of claim 1, wherein responsive to the change in the pressure of the fluid in the reservoir, the relief valve is configured to modify the second flow of fluid from the reservoir such that the change in the first pressure does not exceed a predetermined threshold.

11. A dialysis machine, the dialysis machine comprising:
an actuator, the actuator configured to move a contaminated fluid;
a dialyzer fluidly coupled with the actuator, the dialyzer configured to receive the contaminated fluid and treat the contaminated fluid with a dialysate solution, wherein the dialysate solution is a solution formed via mixing a fluid from a treatment system; and
the treatment system, comprising:
a pump;
a purification device fluidly coupled with the pump, the purification device configured to receive fluid from the pump and treat the fluid;
a reservoir fluidly coupled with the purification device, the reservoir configured to house fluid in the reservoir at a first pressure;
an outlet tube fluidly coupled with the reservoir, the outlet tube configured to facilitate a first flow of fluid from the reservoir to the dialysis machine; and
a relief valve fluidly coupled with the reservoir, the relief valve configured to control a second flow of fluid from the reservoir,
wherein responsive to a change in the first pressure of the fluid in the reservoir, the relief valve is configured to modify the second flow of fluid from the reservoir.

12. The dialysis machine of claim 11, wherein the change in the first pressure of the fluid in the reservoir is responsive to an increase in the first flow of fluid from the reservoir.

13. The dialysis machine of claim 11, wherein the change in the first pressure of the fluid in the reservoir is a decrease in the first pressure of the fluid.

14. The dialysis machine of claim 11, wherein responsive to the change in the first pressure of the fluid in the reservoir, the relief valve is configured to decrease the second flow of fluid from the reservoir.

15. The dialysis machine of claim 14, wherein the relief valve is configured to decrease the second flow of fluid from the reservoir by an amount equal to an amount of the first flow of fluid from the reservoir.

16. The dialysis machine of claim 11, wherein responsive to the change in the first pressure of the fluid in the reservoir, the relief valve is configured to modify the second flow of fluid from the reservoir to maintain a second pressure of the fluid in the reservoir.

17. A system, the system comprising:
an inlet valve, the inlet valve configured to receive a fluid and control a first flow of fluid;
a pump fluidly coupled with the inlet valve, the pump configured to receive the first flow of fluid from the inlet valve and pump the fluid;
a purification device fluidly coupled with the pump, the purification device configured to receive fluid from the pump and treat the fluid;
a reservoir fluidly coupled with the purification device, the reservoir configured to house fluid in the reservoir at a first pressure;
a reservoir gauge coupled with the reservoir, the reservoir gauge configured to determine pressure of the fluid in the reservoir; and
a relief valve fluidly coupled with the reservoir, the relief valve configured to control a second flow of fluid from the reservoir,
wherein responsive to the reservoir gauge determining the pressure of the fluid in the reservoir is the first pressure, the inlet valve is configured to transition to a closed state and restrict the first flow of fluid to the pump.

18. The system of claim 17, wherein the pump is configured to pump the fluid at a constant flow rate.

19. The system of claim 17, further comprising a flowmeter communicably coupled with the pump, wherein the flowmeter is configured to determine a flow rate of fluid from the pump.

20. The system of claim 17, further comprising a feedback tube fluidly coupled with the relief valve and an inlet tube in communication with the pump, wherein the feedback tube is configured to facilitate movement of fluid from the relief valve to the pump.
